(19) United States
(12) Patent Application Publication
(10) Pub. No.: US 2013/0157375 A1
(43) Pub. Date: Jun. 20, 2013

(54) COMPOUND

(71) Applicants: Robert Hider, London (GB); Yongmin Ma, London (GB); Ulrich Schaible, Borstel (DE); Maria Podinovskaia, Ithaca, NY (US)

(72) Inventors: Robert Hider, London (GB); Yongmin Ma, London (GB); Ulrich Schaible, Borstel (DE); Maria Podinovskaia, Ithaca, NY (US)

(73) Assignees: LONDON SCHOOL OF HYGIENE AND TROPICAL MEDICINE, London (GB); KING'S COLLEGE LONDON, London (GB)

(21) Appl. No.: 13/643,198

(22) PCT Filed: Apr. 27, 2011
(86) PCT No.: PCT/GB11/50835

§ 371 Date: Mar. 6, 2013

(30) Foreign Application Priority Data
Apr. 29, 2010 (GB) 1007209.8

(51) Int. Cl. C07D 213/68; C07D 405/14; C07D 401/14; C07D 405/12; C07D 401/12

(52) U.S. Cl. CPC C07D 213/68; C07D 405/12; C07D 401/12; C07D 401/14; C07D 405/14
USPC 436/84; 546/300; 546/283.1; 546/278.7; 546/256

(57) ABSTRACT

In one aspect, there is provided a fluorescent iron-binding compound bound to a solid phase. Also provided is a method for detecting non-transferrin bound iron in a sample, comprising contacting the sample with a fluorescent iron-binding compound bound to a solid phase and detecting a fluorescent signal derived from the fluorescent iron-binding compound bound to the solid phase, wherein the fluorescent signal is indicative of non-transferrin bound iron levels in the sample. Further provided is use of a fluorescent iron-binding compound bound to a solid phase to detect non-transferrin bound iron in a sample.

### FIELD

The present invention relates to the detection of iron levels in a sample, for example to the quantification of non-transferrin bound iron (NTBI) in a biological fluid. The present invention relates to a method for performing such detection, as well as compounds applicable in such a method and uses of such compounds.

### BACKGROUND

Non-transferrin-bound iron (NTBI) is commonly found in the circulation of patients suffering from iron-overload. In healthy individuals, almost all serum iron is bound to the iron-carrier protein, transferrin. However, in iron-overloaded individuals, the iron binding capacity of transferrin in the serum is insufficient to bind all available iron. The resulting excess iron may bind to other proteins or molecules in the serum, and is referred to as NTBI.

NTBI may result from iron overload due to various diseases and their treatments, for example: repeated transfusions, e.g. in order to treat hemolytic diseases, hemoglobinopathics (such as thalassemia patients) or other fauns of anemia whose treatment demands blood transfusions and/or iron infusion (e.g. dialysis patients); an inherited defect causing excess iron absorption, e.g. hereditary hemachromatosis; treatments resulting in haemoglobin catabolism, e.g. chemotherapy and heart bypass operations; and following treatment for anemia with erythropoietin and intravenous iron supplements, e.g. in dialysis patients. It is estimated world wide that there are 500,000 transfusion dependent thalassaemia patients, 300,000 sickle cell anemia patients and 20,000 bone marrow transplants per annum, all of whom may be at risk of developing iron overload and NTBI.

The nature of NTBI is uncertain but there is good evidence for it to be a variable mixture of mononuclear iron(III) citrate complexes, oligonuclear iron(III) citrate complexes and iron-albumin complexes (Biochemica et Biophysica Acta 2009, 1794:1449-1458). The major toxicity associated with NTBI is that the associated iron is not only directed to cells which express the transferrin receptor. Instead, NTBI delivers iron to highly vascular tissue such as the heart and endocrine organs. Iron accumulation in these organs leads to a wide range of disease states, for instance, diabetes and heart diseases. It is therefore important clinically to control the level of NTBI in subjects suffering from iron overload.

Iron-overload is commonly treated by the use of iron chelating agents. However, the accurate monitoring and quantification of NTBI is critical for the use of such agents (see e.g. Blood (2000), 96:3707-3711). There is therefore a need for simple, fast and inexpensive methods for detecting NTBI in large numbers of samples derived from subjects at risk of iron overload.

Known methods for the detection of iron overload include determination of total serum iron via chemical/physicochemical methods, determination of the percent transferrin-iron saturation or serum iron-binding capacity, by measuring high-affinity binding of radioactive iron to serum components or by determining circulating ferritin levels by immunoassay. These methods all suffer from a lack of sensitivity to the detection of low to moderate iron overload and are not specific for NTBI.

A method which specifically determines NTBI in a biological sample is disclosed in Anal. Biochem. (1990) 186:320-323. This method utilizes an iron chelator such as deferriprone which forms a colored complex when in contact with sample NTBI. However, this method is based on the use of HPLC and is therefore labour intensive and not suited to high-throughput screening.

Methods Enzymol. (1994) 233, 82-89 discloses a method in which bleomycin binds to NTBI, but not to transferrin-bound iron, resulting in DNA cleavage products which are quantified using thiobarbituric acid. This method, however, may underestimate NTBI, by only monitoring redox active NTBI and may give false negative results, limiting its clinical usefulness.

A number of methods use an iron scavenging molecule, for instance nitrilotriacetic acid (NTA) or oxalate, in order to detect the presence of NTBI. However, this leads to the introduction of an additional manipulation step into the assay, typically ultrafiltration. As well as increasing the cost of the method, such steps render the method less adaptable to automated procedures.

WO 00/36422 discloses a method in which a sample is incubated with a surface coated with a polymer-conjugated faint of an iron chelator, such as a desferrioxamine (DFO) polymer, such that NTBI is captured by the iron chelator. A solution of a labelled moiety containing bound iron is then added. If the iron chelator has not already been saturated by NTBI, it can capture iron from the labelled moiety resulting in a change in signal. However, a disadvantage of this method is that it is labour-intensive due the present of multiple assay steps, and not well-suited to large scale use.

WO 2004/04052 discloses conjugates comprising an iron-chelating group and a fluorescent label and their use in detecting NTBI. These conjugates bind NTBI in a solution containing the sample and produce a detectable signal related to NTBI levels. However, a problem with fluorescent-based methods such as this is that they suffer from interference from the highly variable absorption and autofluorescence properties of serum samples in the UV and visible regions of the spectrum. Thus reliable measurements cannot be obtained in the presence of serum proteins. This limits the accuracy and sensitivity of such methods, particularly at relatively low to moderate NTBI levels.

None of the currently known methods for determining NTBI have proved to be sufficiently reliable for routine clinical application (see Anal. Biochem. (2005) 341:241-250). Accordingly, there is still a need for a reliable assay and reagents for the measurement of non-transferrin-bound iron (NTBI). Such a method would greatly assist the management of patients who receive chelation therapy, for instance, thalassaemia, sickle cell anaemia, myelodysplasia syndrome (MDS) and haemochromatosis patients. A suitable assay could be used to diagnose iron overload in all these patient classes and to provide an aid to assess the efficacy of chelation therapy.

### SUMMARY

Accordingly, in one aspect the present invention provides a fluorescent iron-binding compound bound to a solid phase.

In one embodiment, the compound comprises an iron-binding moiety and a fluorophore. Preferably a fluorescent signal generated by the fluorophore is modulated in response to binding of iron to the iron-binding moiety.

In one embodiment, the compound comprises a group of formula I:

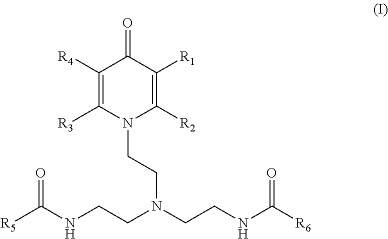

wherein R₁, R₂, R₃ and R₄ are each independently selected from hydrogen, hydroxyl and C₁-C₅ alkyl, e.g. methyl, provided that at least one of R₁ and R₄ is hydroxyl; R₅ comprises a linkage to a solid phase; and R₆ comprises a fluorophore.

Preferably the fluorophore is selected from the group consisting of fluorescein, rhodamine, dansyl, phycoerythrin, phycocyanin, allophycocyanin, o-phthaldehyde, cascade blue, coumarin, naphthalenes, pyrenes, pyridyloxazole derivatives and fluorescamine. Most preferably the fluorophore comprises fluorescein.

In one embodiment, the compound is covalently bound to the solid phase. For example, the compound may be linked to a solid phase by a group of formula Ia:

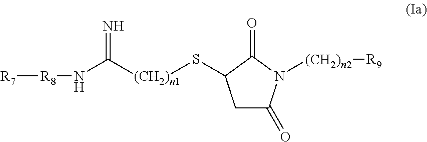

wherein R₇ comprises a solid phase; R₈ is optionally present and comprises a protein, e.g. albumin; R₉ comprises a bond to the compound, and n1 and n2 are each independently an integer between 1 and 5.

In one embodiment, the solid phase comprises beads or microspheres.

In a further aspect, the present invention provides a method for detecting non-transferrin bound iron in a sample, comprising contacting the sample with a fluorescent iron-binding compound bound to a solid phase; and detecting a fluorescent signal derived from the fluorescent iron-binding compound bound to the solid phase, wherein the fluorescent signal is indicative of non-transferrin bound iron levels in the sample.

In one embodiment, the fluorescent signal is detected by flow cytometry. Preferably the method provides a fluorescent signal indicative of iron levels at least in the concentration range 0.01 to 1 μM.

In a further aspect, the present invention provides a method for monitoring iron overload in a subject, comprising detecting non-transferrin bound iron in a sample from the subject by a method as defined above.

In a further aspect, the present invention provides use of a fluorescent iron-binding compound bound to a solid phase to detect non-transferrin bound iron in a sample.

In a further aspect, the present invention provides a kit for detecting non-transferrin bound iron in a sample, comprising a fluorescent iron-binding compound bound to a solid phase, packaged in one or more containers with one or more further reagents, and optionally instructions for performing a method as defined above.

In a further aspect, the present invention provides a compound according to formula (II):

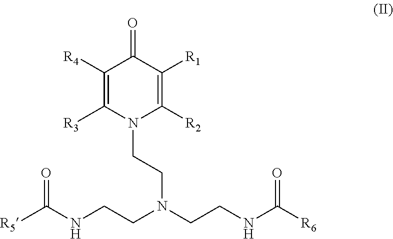

wherein R₁, R₂, R₃ and R₄ are each independently selected from hydrogen, hydroxyl and C₁-C₅ alkyl, e.g. methyl, provided that at least one of R₁ and R₄ is hydroxyl; R₅′ comprises a group capable of forming a linkage to a solid phase; and R₆ comprises a fluorophore.

In a further aspect, the present invention provides a method for producing a compound of formula (I) comprising reacting a compound of formula (II) with a solid phase, wherein formula (I) and formula (II) are as defined above.

### BRIEF DESCRIPTION OF DRAWINGS

FIG. 1 shows a diagrammatic representation of a fluorescent iron-binding compound according to one embodiment of the present invention. A—chelation unit for iron scavenging. B—fluorescence unit for signalling. C—linkage to solid phase. D—bound iron. E—solid phase.

FIG. 2 shows a reaction scheme for the synthesis of a fluorescent iron binding compound (CP805).

FIG. 3 shows a reaction scheme for the linkage of a fluorescent iron binding compound to a solid phase.

FIG. 4 shows uniform labelling of beads with a fluorescent iron sensor and their response to iron. Sensor-labelled beads were detected by confocal microscopy at low (A, C) and high (B, D) magnification, in iron-free PBS buffer (A, B) or in 190 mM ferric ammonium citrate (FeCi).

FIG. 5 shows uniform labelling of beads with fluorescent iron sensor and their response to iron. Scatter plot of beads detected by flow cytometry. The gated homogenous population was chosen for fluorescence analysis. At least 10,000 events were collected. Overlay of histograms shows background autofluorescence of unlabelled beads (grey) and high and uniform fluorescence of sensor-labelled beads in the presence of 0-100 μM Fe-NTA at the molar ratio of 1:2.3.

FIG. 6 shows fluorescence of sensor-labelled beads (SLB) in response to iron in presence of serum. Points show median fluorescence of SLB titrated with iron-NTA with subsequent addition of human serum. Iron/NTA was added at 1:2 molar ratio, at final concentrations of iron of 0.001, 0.005, 0.01, 0.05, 0.1, 0.5, 1, 5, 10 and 100 μM. Human serum was added at 10% (v/v) final concentration. Samples were fixed with paraformaldehyde at final concentration of 2% (w/v). Fluorescence of SLB was analysed by flow cytometry as described for FIG. 5. Median fluorescence was calculated based on at least 10,000 events and adjusted for background fluorescence of unlabelled beads. Median fluorescence values are plotted as mean±SEM of three experiments each using a different donor.

FIG. 7 shows examples of tripyridinone (1,2) and tripyrone (3) iron chelators. In each case, R may represent a fluorophore and/or a linkage to a solid phase.

FIG. 8 shows the synthesis of bidentate hydroxypyridinone.

FIG. 9 shows the synthesis of bidentate hydroxypyranone.

FIG. 10 shows the synthesis of hexadentate hydroxypyridinone.

FIG. 11 shows the synthesis of hexadentate hydroxypyranone.

FIG. 12 shows the general structure of chelator-labeled fluorescent beads.

FIG. 13 shows a standard curve of fluorescence against iron concentration for fluorescent beads conjugated with bidentate hydroxypyridinone.

FIG. 14 shows a standard curve of fluorescence against iron concentration for fluorescent beads conjugated with hexadentate hydroxypyridinone.

FIG. 15 shows a standard curve of fluorescence against iron concentration for fluorescent beads conjugated with bidentate hydroxypyranone.

FIG. 16 shows a standard curve of fluorescence against iron concentration for fluorescent beads conjugated with hexadentate hydroxypyranone.

FIG. 17 shows a standard curve of fluorescence against iron concentration for fluorescent beads conjugated with CP805 via albumin.

FIG. 18 shows the results of kinetic studies of fluorescent beads conjugated with hexadentate hydroxypyridinone with iron-citrate, iron-citrate-albumin and apo-transferrin.

FIG. 19 shows a comparison of two methods for NTBI measurement, using (a) fluorescent beads conjugated with hexadentate hydroxypyridinone and (b) a standard NTA assay.

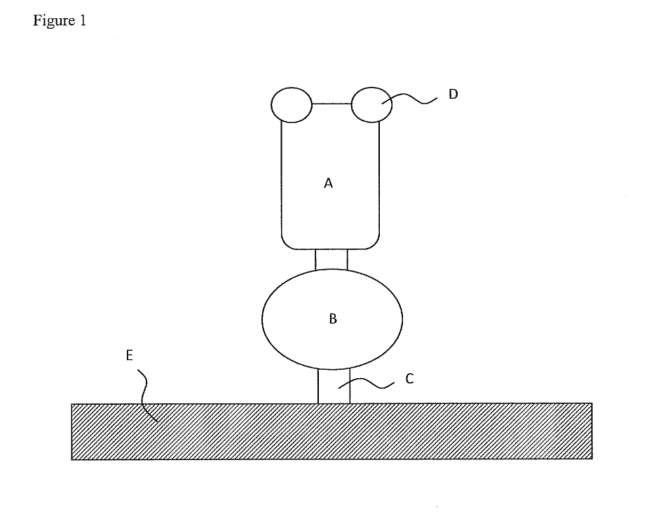

### DETAILED DESCRIPTION

In one aspect, the present invention relates to a fluorescent iron-binding compound bound to a solid phase. In some embodiments, the invention may relate to a solid phase which comprises the fluorescent iron-binding compound, or a solid phase which has been derivatized with the fluorescent iron-binding compound.

By “fluorescent iron-binding compound” it is typically meant any compound which is capable of (a) generating a detectable fluorescent signal and (b) binding iron. Preferably the fluorescent signal generated by the compound is responsive to iron binding by the compound.

One of the properties of ionic iron is its inherent ability to affect the fluorescence properties of fluorophores when in atomic or molecular contact, usually resulting in the quenching of the fluorescence signal (see Lakowicz, J-R. (1983) Principles of Fluorescence Spectroscopy, Plenum Press, New York, pp. 266 ff.).

In one embodiment, the compound comprises an iron-binding moiety and a fluorophore. Preferably a fluorescent signal generated by the fluorophore is modulated in response to binding of iron to the iron-binding moiety. For example, in one embodiment the intensity of the fluorescent signal generated by the fluorophore is related to the amount of the iron which is bound to the iron-binding moiety. In one embodiment, the intensity of the signal is stoichiometrically related to the iron bound by the iron binding moiety. Preferably the fluorescent signal and iron binding are inversely related, i.e. binding of iron to the iron-binding moiety reduces the intensity of the fluorescent signal.

Iron-binding compounds are well known in the art and the iron binding moiety used in the present invention may be based on any such compound, including synthetic and natural organic compounds such as proteins.

In particular embodiments, the iron-binding moiety may comprise an iron-binding protein. Suitable iron-binding proteins include transferrin, apo-transferrin, lactoferrin, ovotransferrin, p97-melanotransferrin, ferritin, ferric uptake repressor (FUR) protein, calcineurin, acid phosphatase and ferredoxin.

In one embodiment the iron-binding moiety comprises an iron chelator. The iron chelator may bind to or combine with iron ions to form a chelate complex comprising a central iron ion. Typically the iron chelator is a polydentate ligand which forms multiple bonds with the iron ion. The iron chelator typically comprises non-metal atoms, two or more of which atoms are capable of forming a bond with the iron ion.

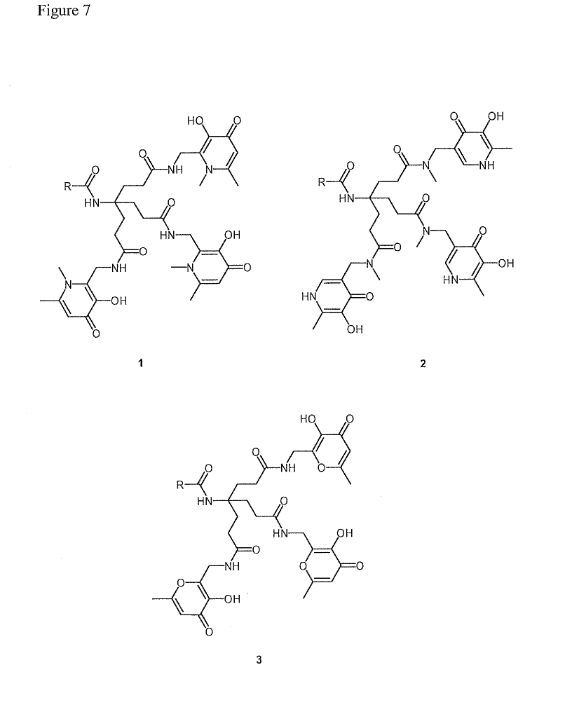

In particular embodiments, the iron chelator may comprise desferrioxamine, phenanthroline, ethylene diamine tetra-acetic acid (EDTA), diethylene triamine-pentaacetic acid (DTPA) or N,N′-bis(2-hydroxybenzoyl)ethylenediamine-N,N′-diacetic acid (HBED). Further iron chelators suitable for use in the present invention are disclosed in U.S. Pat. Nos. 4,840,958, 5,480,894, 4,585,780, 5,925,318 and in Hider (1996) Acta Haematologica 95:6-12. In specific embodiments, the compound comprises a tripyridinone or tripyrone iron chelator, e.g. having a structure shown in FIG. 7 (see Ma Y M and Hider R C, Bioorg Med Chem. 2009, 17(23), 8093-8101).

The compound preferably comprises a fluorophore which can be quantified via its fluorescence. The term “fluorophore” is used to describe a functional group in the compound that fluoresces. The fluorophore typically allows the generation of a direct correlation between changes in fluorescence and iron-binding to the compound, which can in turn be related to NTBI concentration in a sample. Fluorescence may be detected following application of a suitable excitatory light.

Fluorophores are well known and used extensively in other biological applications such as immunochemistry. Common fluorophores include fluorescein and its derivatives, rhodamine and derivatives, dansyl, phycoerythrin, phycocyanin, allophycocyanin, o-phthaldehyde, cascade blue, coumarin and its derivatives, naphthalenes, pyrenes and pyridyloxazole derivatives and fluorescamine. See, for example, Haugland, Handbook of Fluorescent Probes and Research Chemicals, Sixth Ed., Molecular Probes, Eugene, Oreg., 1996. In particular embodiments, the fluorophore may comprise (absorption/emission wavelengths in nm in brackets): Alexa Fluor 350 (346/442), Marina Blue (365/460), Fluorescein-EX (494/518) FITC (494/518), calcein (485/517), tetramethylrhodamine (555/580), rhodamine Red-X (570/590), Texas Red-X (595/615) or Lucifer Yellow (425/531). Alternatively, the fluorophore may comprise a fluorescent protein such as green fluorescent protein, yellow fluorescent protein, cyan fluorescent protein or red fluorescent protein, or a derivative thereof.

In general, the iron-binding moiety, fluorophore and solid phase (e.g. beads) may be linked in the compound using any suitable linkages. For example, in one embodiment, each of the iron-binding moiety, fluorophore and solid phase is conjugated to a linker molecule comprising at least 3 functional groups (e.g. amino or carboxyl groups). Suitable linkers in this embodiment include norspermidine (Bergeron R J, Acc Chem Res. 1986, 19, 105-113) and tris(2-aminoethyl)amine, each of which contains three amino groups; lysine which contains two amino groups and one carboxyl; 5-aminobenzene-1,3-dioic acid (Zhou T et al. J Med Chem. 2006, 49, 4171-4182) which has one amino group and two carboxyl groups; and nitrilotriacetic acid (NTA) which has three carboxyl groups. The structures of these linkers are well known in the art and can be made using known techniques.

In a preferred embodiment, the compound or iron-binding moiety thereof comprises a group of formula (I):

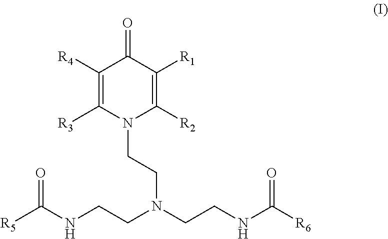

wherein:
R₁, R₂, R₃ and R₄ are each independently selected from hydrogen, hydroxyl and C₁-C₅ alkyl, e.g. methyl, provided that at least one of R₁ and R₄ is hydroxyl;
R₅ comprises a linkage to a solid phase; and
R₆ comprises a fluorophore.

In one embodiment, R₁ is hydroxyl, R₂ is methyl and R₃ and R₄ are hydrogen. R₅ may, for example, comprise a direct bond to the solid phase or an indirect linkage to the solid phase via any suitable linker, e.g. a linker as described above. Thus R₅ may comprise, for example, a straight or branched alkyl or alkenyl chain optionally substituted with one or more reactive functional groups such as carbonyl, carboxyl, amino, imino, hydroxyl, sulfhydryl, maleimido and so on, and may optionally further comprise one or more ether or thioether groups. Preferably R₅ comprises a group of formula (Ia):

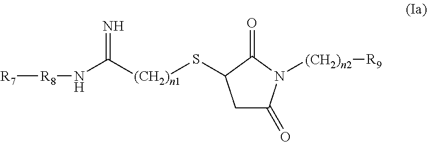

wherein R₇ comprises a solid phase; R₈ is optionally present and comprises a polypeptide or protein, e.g. albumin; R₉ comprises a bond to the group of formula (I), and n1 and n2 are each independently an integer between 1 and 5. By optionally present it is meant that R₈ may be present or absent, i.e. where present R₈ comprises a polypeptide or protein, and where absent R₈ represents a bond between R₇ and an N atom.

In one embodiment the fluorophore is fluorescein, i.e. R₆ comprises a group of formula (IIb):

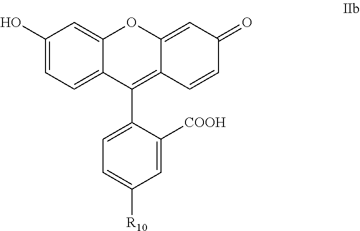

wherein R₁₀ comprises a bond to the group of formula (I).
R₇ may optionally further comprise an alkyl linker (e.g. C₁-C₅) to the solid phase. Preferably n1 is 3 and n2 is 2.

In further embodiments, the compound comprises fluoresceinated deferrioxamine (Fl-DFO) or 5-4,6-dichlorotriazinyl aminofluorescein (DCTF)-apo-transferrin (Fl-aTf). In another embodiment, the compound comprises a chimeric protein, for example a protein fluorophore (e.g., GFP) linked to an iron binding protein. Chimeric proteins can be produced via well known recombinant techniques.

In a further embodiment, the compound comprises a group of formula VIII:

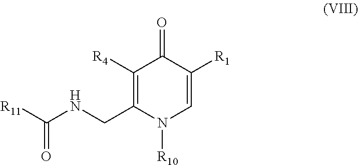

wherein R₁, R₂ and R₄ are each independently selected from hydrogen, hydroxyl and C₁-C₅ alkyl, e.g. methyl, provided that at least one of R₁ and R₄ is hydroxyl; R₁₀ is hydrogen or C₁-C₅ alkyl, e.g. methyl; and R₁₁ comprises a fluorophore and a linkage to a solid phase. Preferably R₁ is hydrogen, R₂ is methyl, R₄ is hydroxyl and R₁₀ is methyl.

In one embodiment, the compound may comprise a hydroxypyridinone chelator, e.g. a tripyridinone or a bidentate or hexadentate pyridinone group as shown in FIG. 7, 8 or 10. For instance, the compound may comprise a group of formula IX:

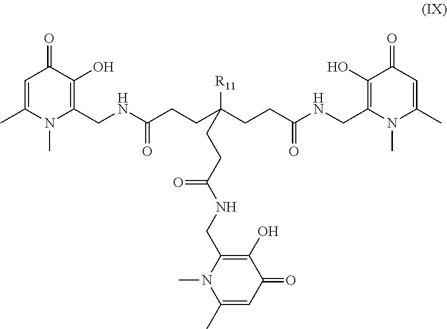

wherein R₁₁ comprises a fluorophore and a linkage to a solid phase.

In a further embodiment, the compound comprises a group of formula XI:

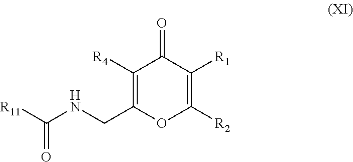

wherein R₁, R₂ and R₄ are each independently selected from hydrogen, hydroxyl and C₁-C₅ alkyl, e.g. methyl, provided that at least one of R₁ and R₄ is hydroxyl; and R₁₁ comprises a fluorophore and a linkage to a solid phase. Preferably R₁ is hydrogen, R₂ is methyl, and R₄ is hydroxyl.

In one embodiment, the compound may comprise a hydroxypyranone chelator, e.g. a tripyranone or a bidentate or hexadentate pyranone group as shown in FIG. 7, 9 or 11. For instance, the compound may comprise a group of formula XII:

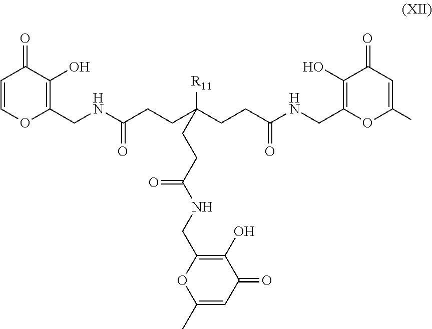

wherein R₁₁ comprises a fluorophore and a linkage to a solid phase.

In one embodiment, R₁₁ comprises a group of formula X:

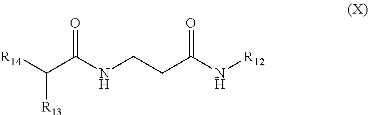

wherein R₁₂ comprises a bond to the compound of formula IX or formula XII;
R₁₃ comprises a fluorophore, e.g. fluorescein or Alexa Fluor 488; and
R₁₄ comprises a linkage to a solid phase.

The nature of the solid phase to which the fluorescent iron-binding compound is linked is not particularly limited, i.e. the solid phase can be made of any insoluble or solid material. For example, suitable solid phases may be comprised of agarose, silicon, rubber, glass, glass fiber, silica gel, cellulose, metal (e.g. steel, gold, silver, aluminum, silicon and copper), or polymers (e.g. polystyrene, polycarbonate, polyethylene, polypropylene, polyamide, polyacrylamide, polyvinylidenedifluoride).

Preferably the solid phase is a particulate material, wherein the particles may have any shape and dimensions. Typically the particles have at least one dimension of 100 mm or less, 50 mm or less, 10 mm or less, 1 mm or less, 100 μm or less, 50 μm or less. For example, the particles may have a diameter of about 1 to 100 μm, e.g. 1 to 20 μm. Typically the solid phase comprises beads which are predominantly spherical in form. The beads may comprise microspheres, e.g. particles with a diameter of 1 to 10 microns. Alternative solid phases to beads include flat surfaces, such as the walls of a reaction vessel. For example, the solid phase may be an assay plate such as those used in ELISA assays, e.g. a 96-well plastic microtiter plate.

Suitable beads may be comprised of silica gel, glass (e.g. controlled-pore glass (CPG)), nylon, Sephadex, Sepharose, cellulose, or a metal or plastic. Beads can be swellable, e.g., polymeric beads such as Wang resin, or non-swellable (e.g., CPG). In a preferred embodiment, the beads are magnetic, i.e. contain superparamagnetic crystals as described in U.S. Pat. No. 4,654,267, e.g. Dynabeads® obtainable from Invitrogen Corp., Carlsbad, Calif. Other commercially available microspheres can be obtained from 3M Scotchlite Glass Bubbles, Biosphere Medical (Rockland, Mass.), Luminex microspheres (Austin, Tex.), Spherotech, Inc. (Libertyville, Ill.) and Structure Probe, Inc. (West Chester, Pa.).

The compounds of the present invention can typically be synthesized using well known chemical synthesis procedures.

For example, amine-reactive fluorophores (typically a fluorophore modified with a reactive group such as dichlorotriazinyl, isothiocyanate, succinimidyl ester, sulfonyl chloride and the like) can be reacted with an iron-binding compound containing an amino group, or with an amine-containing linker molecule to which the iron-binding compound is also conjugated. Amine-reactive fluorophores are mostly acylating reagents, which form carboxamides, sulfonamides, ureas or thioureas upon reaction with amines. Iron-binding proteins and chelators such as desferrioxamine typically contain one or more amino group and can therefore be conveniently reacted with activated fluorophores. Following conjugation, unconjugated fluorophore is removed, usually by gel filtration, dialysis, HPLC or a combination of these techniques. Methods for conjugating a fluorophore to an iron-binding compound are generally described in WO 2004/040252.

Techniques for binding a compound to a solid phase are known. The compound can be directly attached to the solid support by means of reactive groups or alternatively, by means of a coupling agent or linker, e.g. a linker as described above.

For attachment of the solid phase (e.g. beads) to the fluorescent iron-binding compound, the solid phase (e.g. beads) may carry functional groups such as hydroxyl, thiol, carboxyl, aldehyde or amino groups. These may in general be provided, for example, by treating uncoated beads, to provide a surface coating of a polymer carrying one of such functional groups, e.g. polyurethane together with a polyglycol to provide hydroxyl groups, or a cellulose derivative to provide hydroxyl groups, a polymer or copolymer of acrylic acid or methacrylic acid to provide carboxyl groups or an aminoalkylated polymer to provide amino groups. U.S. Pat. No. 4,654,267 describes the introduction of many such surface coatings. Other coated particles may be prepared by modification of the beads according to the U.S. Pat. Nos. 4,336,173, 4,459,378 and 4,654,267.

For example, the amino groups initially present in the beads may be reacted with a diepoxide as described in U.S. Pat. No. 4,654,267 followed by reaction with methacrylic acid to provide a terminal vinyl grouping. Solution copolymerization with methacrylic acid yields a polymeric coating carrying terminal carboxyl groups. Similarly, amino groups can be introduced by reacting a diamine with the above product of the reaction with a diepoxide, while reaction with a hydroxylamine such as aminoglycerol introduces hydroxy groups. In some embodiments, a protein such as albumin may be bound to the solid phase in order to multiply the number of amino groups. Thiol (sulfhydryl) groups may be introduced by reacting an amine with Traut's reagent (2-iminothiolane).

The coupling of a fluorescent iron-binding compound to a solid phase (e.g bead) is typically via a covalent linkage. In some embodiments, the fluorescent iron-binding compound may be linked to the solid phase via a reversible linker, e.g. via a peptide comprising a proteolytic recognition site or a reducible disulfide group. A variety of reversible crosslinking groups can be obtained from Pierce Biotechnology Inc. (Rockford, Ill., USA).

In one embodiment, the solid phase comprises a sulfhydryl group. The fluorescent iron-binding compound may be linked to the solid phase by, for example, reacting a sulfhydryl group on the solid phase with a reactive group on the fluorescent iron-binding compound, e.g a maleimide.

For example, a compound of formula (I) may be synthesized by a method comprising reacting a compound of formula (II) with a solid phase:

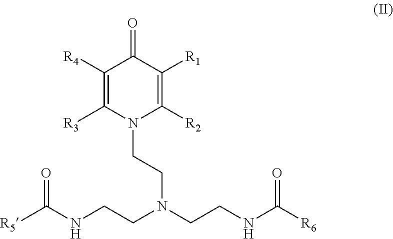

wherein R₁, R₂, R₃ and R₄ are each independently selected from hydrogen, hydroxyl and C₁-C₅ alkyl, e.g. methyl, provided that at least one of R₁ and R₄ is hydroxyl;
R₅′ comprises a group capable of forming a linkage to a solid phase;
and R₆ comprises a fluorophore.
R₅′ may, for example, comprise any suitable functional group capable of reacting with a corresponding functional group present on the solid phase. For example, R₅′ may comprise hydrogen, hydroxyl or straight or branched chain alkyl or alkenyl substituted with one or more reactive functional groups such as carbonyl, carboxyl, amino, imino, hydroxyl, sulfhydryl, maleimido and so on. In one embodiment, R₅′ comprises a group of formula (IIa):

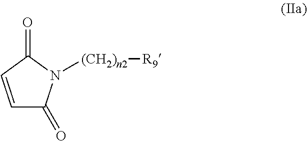

wherein R₉′ comprises a bond to the group of formula (II), and n2 is an integer between 1 and 5, preferably 2.

The solid phase may, for example, comprise a bead comprising a reactive functional group such as a thiol group. In one embodiment, the solid phase comprises a group of formula (III):

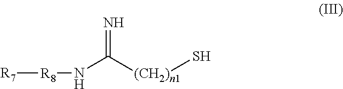

wherein R₇ comprises a solid phase; R₈ is optionally present and comprises a protein, e.g. albumin; and n1 is an integer between 1 and 5, preferably 3. In one embodiment, R₇ may comprise the formula [solid phase]-(CH₂)ₙ₃—, wherein n3 is an integer between 1 and 5, preferably 3.

A solid phase comprising a group of formula (III) may be produced, for example, by reacting an amine-containing solid phase with, e.g. 2-iminothiolane.

A compound of formula (II) may be produced by (a) reacting a compound of formula (IV):

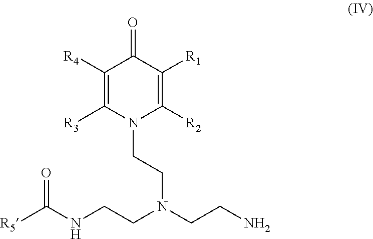

wherein R₁, R₂, R₃ and R₄ are each independently selected from hydrogen, hydroxyl and C₁-C₅ alkyl, e.g. methyl, provided that at least one of R₁ and R₄ is hydroxyl, wherein hydroxyl is optionally protected by a protecting group, e.g. benzyl; and
R₅′ comprises a group capable of forming a linkage to a solid phase;
with a compound of formula (V):

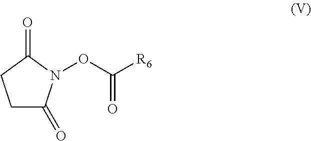

wherein R₆ comprises a fluorophore;
and (b) optionally removing the protecting group.

A compound of formula (V) may be produced by, for example, reacting a carboxyl group in a fluorophore with N-hydroxysuccinimide.

A compound of formula (IV) may be produced by reacting a compound of formula (VI):

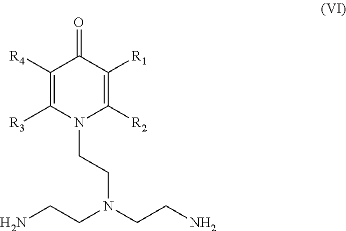

wherein R₁, R₂, R₃ and R₄ are each independently selected from hydrogen, hydroxyl and C₁-C₅ alkyl, e.g. methyl, provided that at least one of R₁ and R₄ is hydroxyl, wherein hydroxyl is optionally protected by a protecting group, e.g. benzyl;
with a compound of formula (VII):

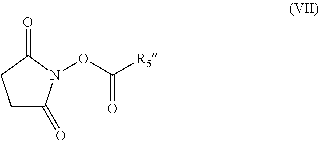

wherein R₅″ comprises a group capable of forming a linkage to a solid phase, e.g. a group of formula (VIIa):

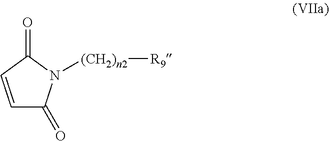

wherein R₉″ comprises a bond to the group of formula (VII), and n2 is an integer between 1 and 5, preferably 2.

Appropriate starting materials (e.g. an amine-containing solid phase, fluorophores and compounds of formulae (VI), (VII) and (VIIa)) are known in the art or can be produced by a skilled person using methods analogous to those described herein, e.g. with reference to Example 1 below.

Once the fluorescent iron-binding compound has been prepared (and optionally bound to a solid phase such as beads), it is preferable to store and further utilise the compound or beads in a plastic container or vessel. For example, the compound or beads may be stored or used in a polypropylene container or vessel, e.g. BD Falcon tubes. It is possible that if kept in a glass container, the compound or beads may lose fluorescence sensitivity, due to the presence of small amounts of iron contamination associated with the glass surface. Storing and using the compound or beads in a plastic container typically avoids iron contamination problems and consequent loss of fluorescence sensitivity.

The fluorescent iron-binding compounds bound to a solid phase as described herein are suitable for use as an indicator of free iron levels in a biological sample. Accordingly, in one embodiment, the compound is used in a method to detect non-transferrin bound iron in a sample, e.g. to quantify the level of NTBI in the sample.

Typically the sample comprises a biological fluid such as blood, serum, plasma, lymph, bile fluid, urine, saliva, sputum, synovial fluid, semen, tears, cerebrospinal fluid, bronchioalveolar lavage fluid, ascites fluid, pus or the like.

The term “free iron levels” may be used to refer to non-transferrin bound iron (i.e. NTBI). NTBI may be used to refer to directly chelatable iron (DCI) which is accessible to exogenous iron chelators; mobilizer-dependent chelatable iron (MDCI) which is accessible to exogenous iron chelators upon addition of mobilizing agents; and labile plasma iron (LPI) including redox active iron, which redox activity is eliminated upon addition of exogenous iron chelators. Mobilizing agents are generally capable of mobilizing immobilized iron which circulates as low molecular weight complexes with compounds such as citrate and phosphate (Grootveld (1989) J. Biol. Chem. 264:4417-4422), in association with amino acids or serum proteins such as albumin, or as microaggregates of iron, which occur due to very low solubility of Fe⁺³ in physiological solutions.

The method may comprise a step of contacting the sample with a fluorescent iron-binding compound bound to a solid phase. Typically the contacting is effected under conditions suitable for binding of the iron-binding moiety of the compound to free iron (i.e. NTBI). The contacting step may be performed at any suitable temperature, preferably at room temperature (e.g. 20 to 25° C.) and at any suitable pH (e.g. 6.0 to 9.0, preferably 7.0 to 8.0, e.g. about 7.4). In one embodiment the buffer comprises nitriloacetic acid.

Fluorescence detection sensitivity may be compromised by background signals, which may originate from endogenous sample constituents (e.g. serum proteins). It has surprisingly been found that interference from such background signals can be greatly reduced by employing a fluorescent iron-binding compound bound to a solid phase. This results in a low, stable background signal which enhances the sensitivity and accuracy of the assay. Thus according to embodiments of the present invention, the method may enable the detection of free iron at concentrations of less than 20 μM, less than 10 μM, less than 1 μM, less than 0.1 μM or less than 0.05 μM. Preferably, the method is capable of quantifying NTBI at least in the concentration range 0.05 to 0.5 μM, more preferably 0.01 to 1 μM, more preferably 0.01 to 5 μM, more preferably 0.01 to 10 μM, more preferably 0.01 to 20 μM.

The fluorescent signal produced by the solid phase may be detected and quantified to determine the levels of free iron in the biological fluid of the subject. Thus the method typically comprises a step of detecting a fluorescent signal derived from the fluorescent iron-binding compound bound to the solid phase, wherein the fluorescent signal is indicative of non-transferrin bound iron levels in the sample. Signal detection may be effected by any suitable instrumentation such as a fluorescent microscope or an ELISA reader.

Preferably the fluorescent signal is detected by a method such as flow cytometry. Flow cytometric methods are well known and are described in numerous references, e.g. Shapiro's Practical Flow Cytometry, Third Edition (Alan R. Liss, Inc. 1995). Flow cytometry can be used to measure fluorescence from the solid phase (e.g. beads) as they pass through a light beam, such as, for example, a laser. Generally, the sample to be analyzed is introduced from a sample tube into or near the center of a faster flowing stream of sheath fluid, which carries the fluid sample to the center of the measuring point in an examination zone (e.g., a flow cell). Thereafter, a continuous wave laser focuses a laser beam on the beads as they pass through the examination zone. Detectors that are optically connected to the examination zone interrogate signal from this zone on one or more detection channels. The detectors may utilize a plurality of detection channels or single-channel detection. Thus the detectors may detect fluorescence emissions from the fluorescent iron-binding molecule bound to a solid phase.

The intensity of signal produced in any of the detection methods described herein may be analyzed manually or using a computer program. Any abnormality in the levels of free iron in the sample is indicative of the presence of a disorder in the subject.

In one embodiment, a look-up table or a standard (calibration) curve is used to equate particular fluorescence readings with a level of NTBI in the sample. A standard curve may be produced by incubating known concentrations of free iron with the fluorescent iron-binding compound bound to a solid phase and obtaining fluorescence values. Fluorescence values from samples containing unknown free iron levels can then be compared to the standard curve to provide an NTBI value.

Another embodiment involves the exclusion of endogenous apo-transferrin and/or iron free transferrin from the sample prior to free iron determination. Apo-transferrin is universally found in human sera, except in cases of extreme iron-overload where the transferrin is 100% iron-saturated. Exclusion of endogenous apo-transferrin can be effected by incubating (i.e., pre-clearing) the sample with anti-apo-transferrin antibodies, such as solid phase coupled anti-transferrin antibodies available from Pharmacia, Uppsala and Bio-Rad Laboratories, Hercules, Calif. Additionally or alternatively anionic beads such as MacroPrep (Registered trademark) High S support beads available from Bio-Rad Laboratories, Hercules, Calif. can be used to exclude apo-transferrin from the sample. In another embodiment, exclusion of apo-transferrin is effected by co-incubating the sample with an apo-transferrin binding metal other than iron such as gallium and cobalt. These metals mimic iron and bind to the indicator molecule of the present invention, preventing their reaction with iron (Breuer and Cabantchik Analytical Biochemistry 299, 194-202 (2001)).

It will be appreciated that the fluorescent iron-binding compound bound to a solid phase can be included in a diagnostic or therapeutic kit. For example, a kit including one or more of the following components described herein (e.g. a fluorescent iron-binding compound bound to a solid phase, and optionally a mobilizing agent), can be packaged in one or more containers with appropriate buffers and preservatives and used for diagnosis or for directing therapeutic treatment.

The compounds disclosed herein bound to a solid phase can be widely used to directly and sensitively detect NTBI which persists in sera of patients, even with low transferrin saturation. Thus, according to still another aspect of the present invention there is provided a method of determining presence or absence of a disorder associated with abnormal levels of free iron in a biological fluid of a subject, comprising the use of a fluorescent iron-binding compound bound to a solid phase.

Examples of disorders and conditions which are associated with abnormal levels of free iron include, but are not limited to, hemolytic diseases hemoglobinopathies, thalassemia, thalassemia major, anemia, sickle cell anemia, aplastic anemia, megaloblastic anemia, myelodyplasia, diseases which require repeated transfusions, diseases which require dialysis, hereditary hemachromatosis, cancer, heart diseases, Megaloblastic Dysplasia Syndrome (MDS) and rheumatoid arthritis.

The subject may be, for example, a mammal. The subject is preferably human. Methods of obtaining body fluids from mammals are well known in the art. It will be appreciated that the source of the fluid may vary depending on the particular disorder which is desired to detect.

Determining iron levels originating from a biological sample of a patient is preferably effected by comparison to a normal sample, which sample is characterized by normal levels of free iron (e.g. no detectable free iron).

The present iron quantification method may also be used in disease management. The method may be used to detect disease in subjects even with low levels of free iron, and to guide a medical practitioner in advising the subject on the type of diet to maintain in terms of iron content and iron availability for adsorption, in order to avoid iron overload. The present method may also be used for large scale screening for free iron overload.

Additional objects, advantages, and features of the present invention will become apparent to a skilled person from the following examples, which are not intended to be limiting.

### EXAMPLES

### Example 1

### Synthesis of CP805

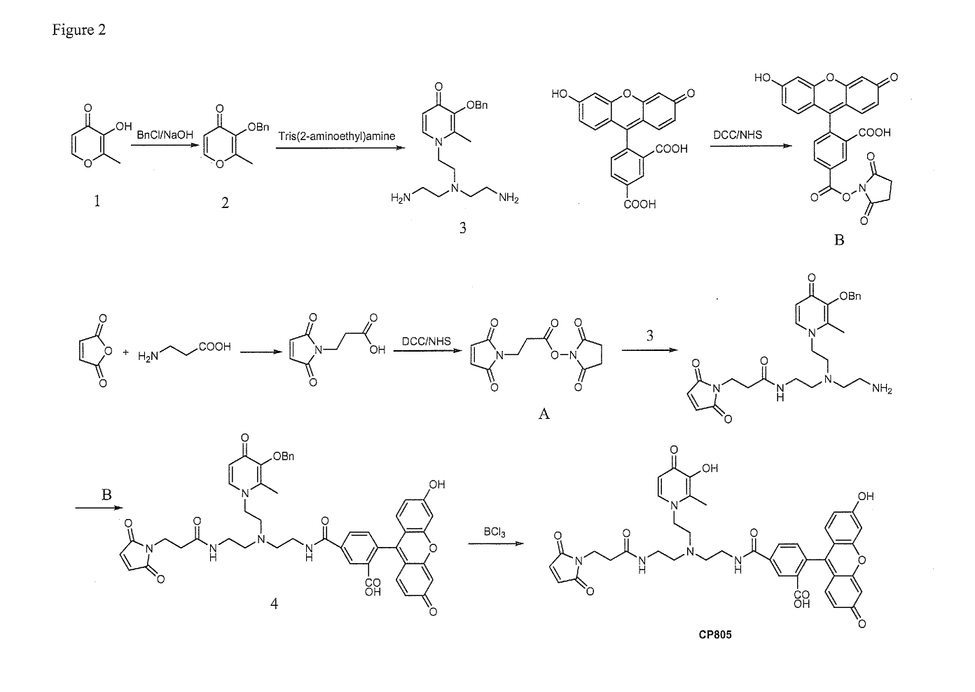

The general methodology adopted for the synthesis of CP805 is shown in FIG. 2. The 3-hydroxyl group of methyl maltol 1 was protected using the benzyl group by reacting with benzyl chloride or benzyl bromide in the presence of sodium hydroxide. The resulting protected maltol 2 was then reacted with tris(2-aminoethyl)amine under mild conditions to afford an oil 3. Compound 3 was then successively reacted with (A) N-hydroxysuccinimide activated 3-maleimidopropanoic acid and (B) N-hydroxysuccinimide activated 5(6)-carboxyfluorescein to produce compound 4. The benzyl protecting group of 4 was then removed by BCl₃ to form CP805.

### 2-Methyl-3-benzyloxypyran-4(1H)-one (2)

To a solution of methyl maltol 1 (63 g, 0.5 mol) in methanol (500 mL) was added sodium hydroxide (22 g, 0.55 mol) dissolved in water (50 mL), and the mixture was heated to reflux. Benzyl chloride (70 g, 0.55 mol) was added dropwise over 30 mins, and the resulting mixture was refluxed overnight. After removal of solvent by rotary evaporation, the residue was mixed with water (200 mL) and extracted with dichloromethane (3×150 mL). The combined extracts were washed with 5% aqueous sodium hydroxide (2×200 mL) followed by water (200 mL). The organic fraction was then dried over anhydrous sodium sulphate, filtered and rotary evaporated to yield an orange oil which solidified on cooling. Recrystallization from diethyl ether gave the pure product 2 as colourless needles (87.5 g, 81%); mp 51-52° C. ¹H-NMR (CDCl₃) δ: 2.12 (s, 3H), 5.11 (s, 2H), 6.25 (d, J=6 Hz, 1H), 7.28 (s, 5H), 7.47 (d, J=6 Hz, 1H).

### 1-{2-[bis(2-Aminoethyl)amino]ethyl}-3-benzoxy-2-methylpyridin-4(1H)-one (3)

To a solution of 2 (2.16 g, 10 mmol) in water:ethanol (50:50 mL) was added tris(2-aminoethyl)amine (2.92 g, 20 mmol). The mixture was heated at 70° C. overnight. After evaporation to reduce the volume to half of the original one, the residue was then extracted with dichloromethane (5×100 mL). The combined organic layers were dried over anhydrous sodium sulphate, filtered, and rotary evaporated to give brown oil (2.2 g, 65%); ¹H-NMR (CDCl₃) δ; 2.05 (s, 3H), 2.35 (m, 10H), 320 (br s, 4H), 3.70 (t, J=7.2 Hz, 2H), 4.84 (s, 2H), 6.02 (d, J=7.2 Hz, 1H), 7.18 (s, 5H), 7.44 (d, J=7.2 Hz, 1H).

Compound 4. Preparation of NHS-Activated 3-Maleimidopropanoic Acid (A):

β-Alanine (0.91 g, 10 mmol) was added to a solution of maleic anhydride (1.0 g, 10 mmol) in 12 ml DMF and stirred for 2 h at room temperature. When the solid dissolved, an ice bath was used to decrease the temperature to 0° C. N-Hydroxysuccinimide (NHS; 1.15 g, 10 mmol) was added into the solution followed by DCC (2.3 g, 12 mmol). After 10 min the ice bath was removed and the reaction was kept at room temperature overnight, which resulted in a white precipitate. The solution was filtered and the precipitate was washed with water (100 ml) and dichloromethane (100 ml). The filtrate was collected and the organic layer was washed with 3×20 ml 5% NaHCO₃ and brine. The organic layer was dried with Na₂SO₄ and dichloromethane was removed under reduced pressure to obtain a white solid (55%) which can be used for next step reaction without further purification.

Preparation of N-Hydroxysuccinimide Activated 5(6)-Carboxyfluorescein (B):

A solution of 5(6)-carboxyfluorescein (1.88 g, 5 mmol) in anhydrous DMF (50 ml) was added DCC (1.15 g, 6 mmol) and NHS (0.58 g, 5 mmol) and the reaction mixture was stirred at room temperature overnight.

Preparation of Compound 4:

The solution containing B was filtered and the filtrate was added to amine 3 (1.72 g, 4 mmol). The reaction mixture was stirred at room temperature overnight, followed by the addition of succinimido 3-maleimidopropanoate (A) prepared above (1.33 g, 5 mmol). The mixture was stirred for 2 days at room temperature. After evaporation to remove DMF, the residue was purified on column chromatography first eluting with MeOH:DCM=1:9 to remove all by-products then increasing the polarity of the eluent to afford compound 4 (1.2 g, 35% yield based on the amine 3). ¹H-NMR (400 MHz, DMSO-d₆) δ: 2.15 and 2.20 (two s, CH3, 3H), 2.59-3.94 (m, CH2, 16H), 4.99 and 5.02 (two s, CH2, 2H), 6.06 (d, J=7.4 Hz, pyridinone 5-CH, 1H), 6.70 (s, maleimide CH, 2H), 7.27-7.40 (m, BnH and pyridinone 6-CH, 6H), 6.20-8.44 (m, fluorescein CH, 9H). ESI-MS: Calculated for C₄₇H₄₃N₅O₁₁ 853.3 (monoisotopic molecular weight M). found 854.2 (M+H)⁺.

Compound CP805.

Compound 4 (1.1 g, 1.3 mmol) was added into CH₂Cl₂ (50 mL) and flushed with nitrogen. After the flask was cooled to 0° C., boron trichloride (1M in CH₂Cl₂, 3 mL) was slowly added and the reaction mixture was allowed to stir at room temperature overnight. The excess BCl₃ was eliminated at the end of the reaction by the addition of methanol (10 mL) and left to stir for another half an hour. After removal of the solvent under reduced pressure, the residue was dissolved in MeOH and precipitated with addition of acetone (3×) to afford the hydrochloric acid salt of CP805 as a yellow solid (0.73 g, 67%). ¹H-NMR (400 MHz, DMSO-d₆) δ: 2.53 and 2.60 (two s, CH3, 3H), 2.35-3.80 (m, CH2, 10H), 4.70-5.10 (m, 6H), 6.50-6.62 (m, fluorescein CH, 4H), 6.74 (s, maleimide CH, 2H), 7.25-8.60 (m, pyridinone CH and fluorescein CH, 7H). ESI-MS: Calculated for C₄₀H₃₇N₅O₁₁ 763.25 (monoisotopic molecular weight M). found 764.07 (M+H)⁺. HRMS: Calculated for C₄₀H₃₈N₅O₁₁ (M+H)⁺764.2568. found 764.2616.

### Example 2

### Synthesis of CP805 Linked to Beads

The linkable iron sensor (compound CP805) was covalently linked to BSA-coated Dynabeads®. These beads were chosen because of ease of handling (recovery of beads by magnetic field) and homogeneous size. Incubation of the beads with BSA led to covalent binding of albumin to the beads via converting the tosyl group to amine. Albumin acted as an amplification step owing to its abundance of amine groups, which were then converted to thiols using Traut's Reagent, and subsequently covalently bound to the iron sensor. This procedure promised a stable coating of beads with a high concentration of the iron sensor and also allowed stoichiometric quantification of the sensor.

Dynabeads® (M-280 tosyl-activated, Invitrogen) were incubated in 1% (w/v) bovine serum albumin (BSA) in PBS on shaker at room temperature for 2 h. The beads were subsequently washed three times with EDTA buffer and incubated with 14 mM Traut's reagent (Pierce Biotechnology, Rockford, USA) in EDTA buffer for an additional 1 h. Following two washes with PBS, the beads were incubated with linkable chelator for a further 1 h, and subsequently washed twice with PBS. Sensor-labelled beads (SLB) were stored at 4° C. in the dark.

### Example 3

### Confocal Microscopy Imaging of CP805 Bound to Beads

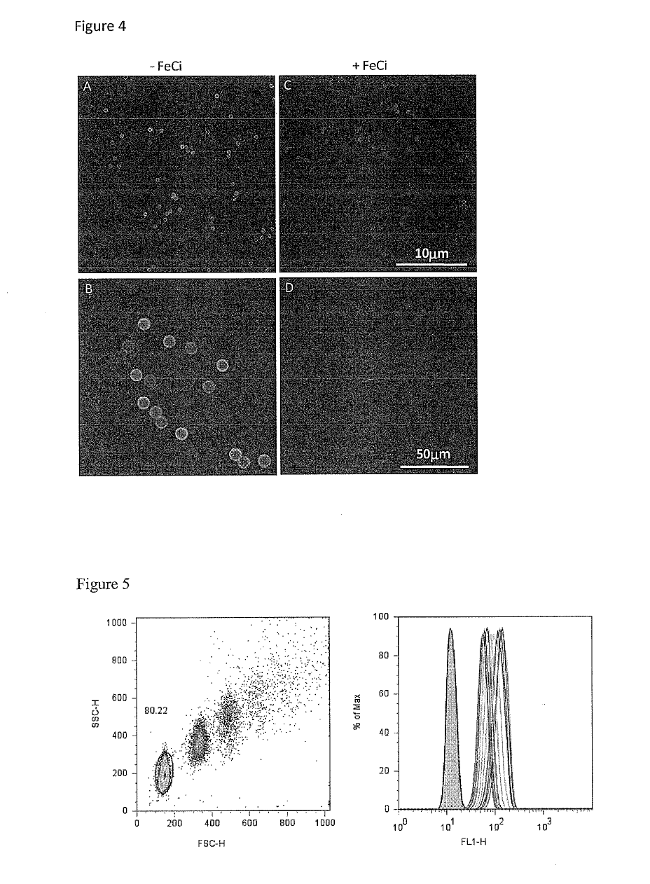

Confocal microscopy imaging confirmed the successful and uniform binding of the iron sensor to the beads and its fluorescence unaffected by the coupling procedure (FIGS. 4, A and B). The fluorescence of the sensor-labelled beads (SLB) was quenched in the presence of iron, confirming their predicted sensitivity to iron (FIGS. 4, C and D). Thus, we have designed fluorescent particles that were sensitive to iron.

To visualise sensor-labelled beads (SLB) inside macrophages, SLB were applied to unlabelled macrophage monolayers for 30 min, washed and further incubated in PBS with or without 0.4 mM iron ammonium citrate, washed and visualised by microscopy.

Fluorescence was visualized using an inverted Zeiss confocal fluorescent microscope and a 63× oil immersion objective. All relevant negative controls were carried out in parallel. Images were acquired by Zeiss LSM Image Browser software, using 488 nm excitation wavelength with a 505-550 nm band-pass filter for emission of green fluorescence signal.

### Example 4

### Detection of CP805 Bound to Beads by Flow Cytometry

Although microscopy provided a quick and straightforward way to assay SLB fluorescence, the plane of focus for each SLB and fluorescence bleaching influenced the intensity of individual SLB fluorescence as detected by confocal microscopy. In order to further improve the reliability and accuracy of detection, fluorescence of SLB was assayed by flow cytometry. The forward-side scatter revealed several populations of events detected by the flow cytometer (FIG. 5). This is most likely due to the tendency of the beads to aggregate into dimers, trimers, strings and stacks as observed in the confocal microscopy images (FIG. 4). To avoid the bead aggregation, the concentration of SLB was kept minimal for flow cytometry analysis, at 0.5-2×10⁵ SLB per 200 μL sample. The major population of events in the forward-side scatter blot most likely represented single beads. We gated on this population for further analysis of SLB fluorescence. The fluorescence of SLB is presented by the histogram in FIG. 5, which reveals a very tight range of fluorescence intensities for individual beads. For comparison, background fluorescence of unlabelled beads is presented by histogram in grey and is much lower than fluorescence of SLB in absence or in presence of iron. Hence, the flow cytometry based analysis of SLB fluorescence provides an excellent means for characterising SLB sensitivity to various conditions and for quantification of iron.

### Example 5

### Flow Cytometric Assay of Free Iron Levels in Serum

Owing to the proved sensitivity of the SLB to fluctuating physiological levels of free iron, we developed a flow cytometric assay based on the SLB to measure free iron levels in serum. The fluorescence of the SLB was assayed in PBS in the presence of healthy human serum samples in an iron titration experiment, with the range of iron concentrations of 0-100 μM.

120 μL quantities of sensor-labelled bead (SLB) suspensions were incubated with 20 μL of nitriloacetic acid (NTA)-buffered iron solutions of known concentrations for 20 min at room temperature, with subsequent addition of 20 μL serum of normal iron status (which does not contain free iron) and 40 μL paraformaldehyde (10% (w/v) PFA in PBS) at a final concentration of 2%. Fixation by paraformaldehyde of serum samples of unknown infection risk is a standard safety requirement in many research and clinical settings. The pH of 7.4 was adjusted for all samples. Measurements were carried out on a FACSCalibur flow cytometer (BD Biosciences, UK). Analysis proceeded via Cell-Quest and FlowJo softwares. Gates were based on dot-plots of untreated bead populations. Median fluorescence of at least 10,000 events were recorded and corrected for bead auto-fluorescence. Standard curve was plotted using GraphPad Prism software, using non-linear regression assuming variable slope sigmoidal dose-response function.

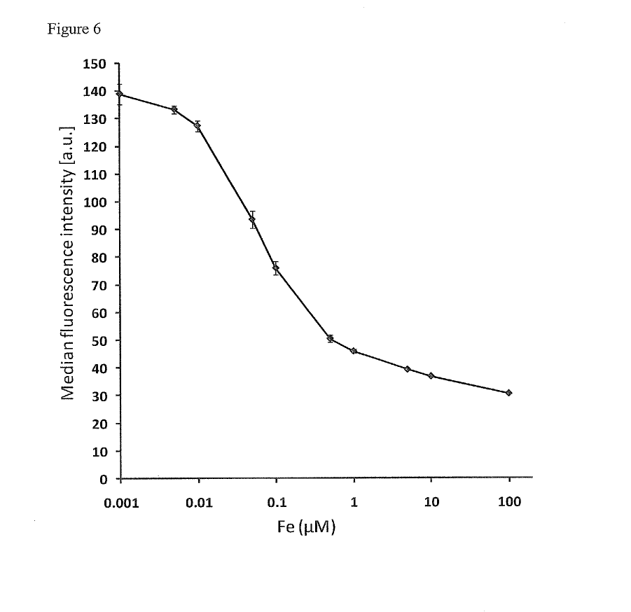

The resulting titration curve is presented in FIG. 6. The fluorescence readings are reproducible between independent serum samples within the same experiment, as reflected by the tight error bars of mean values. This suggests that inter-individual variations in different sera do not affect sensor fluorescence and response to iron. The iron sensor was most responsive to iron concentration range of 0.01-1 μM, for example leading to fluorescence quenching from 89% to 14%, relative to maximal and minimal fluorescence intensities FIG. 6. This implies that the assay would be most sensitive for sera containing free iron at 0.1-10 μM, when applied at 10% concentration for sample analysis.

For serum samples of unknown iron concentrations, 1404 quantities of SLB were incubated with 20 μL of serum samples for 20 min, with subsequent addition of 40 μL paraformaldehyde at a final concentration of 2%, prior to measurement by flow cytometry as described above and estimation of free iron levels with reference to the titration curve.

### Example 6

### Synthesis of Four Iron Chelators

### i) Bidentate Hydroxypyridinone

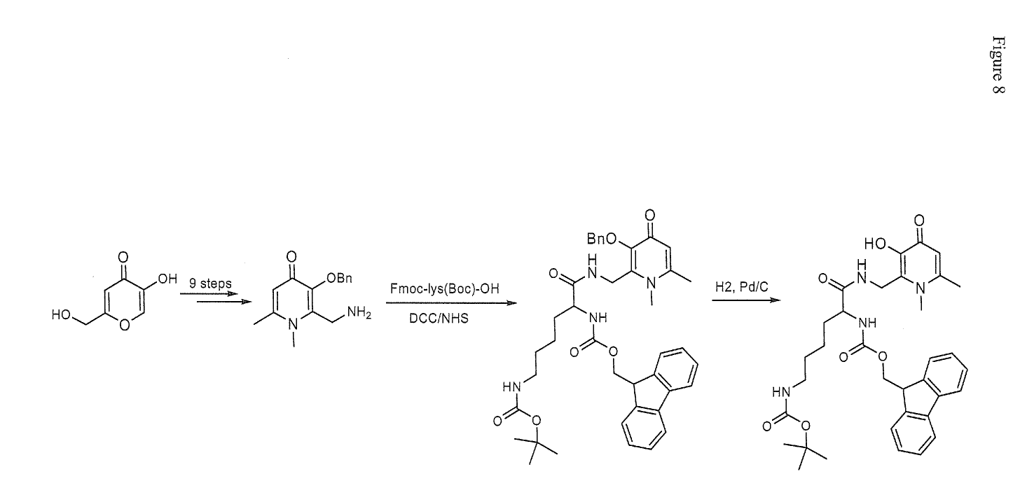

The synthesis of bidentate hydroxypyridinone is shown in FIG. 8. The synthesis of 1,6-dimethyl-2-aminomethyl-3-benzyloxypyridin-4-one which started from kojic acid via 9 steps has been reported previously [Ma Y. et al. J Med Chem. 2004, 47, 6349-6362]. The coupling of the amine with activated Fmoc-lys(Boc)-OH which produced from commercial available Fmoc-lys(Boc)-OH (sigma-aldrich, CAS No. 71989-26-9) with dicyclohexylcarbodiimide and N-hydroxysuccinimide affords the desired protected lysine attached hydroxypyridinone. Deprotection by hydrogenation only removes the benzyl group on the 3-hydroxypyridinone, without influencing the Fmoc and Boc protected amines.

### ii) Bidentate Hydroxypyranone

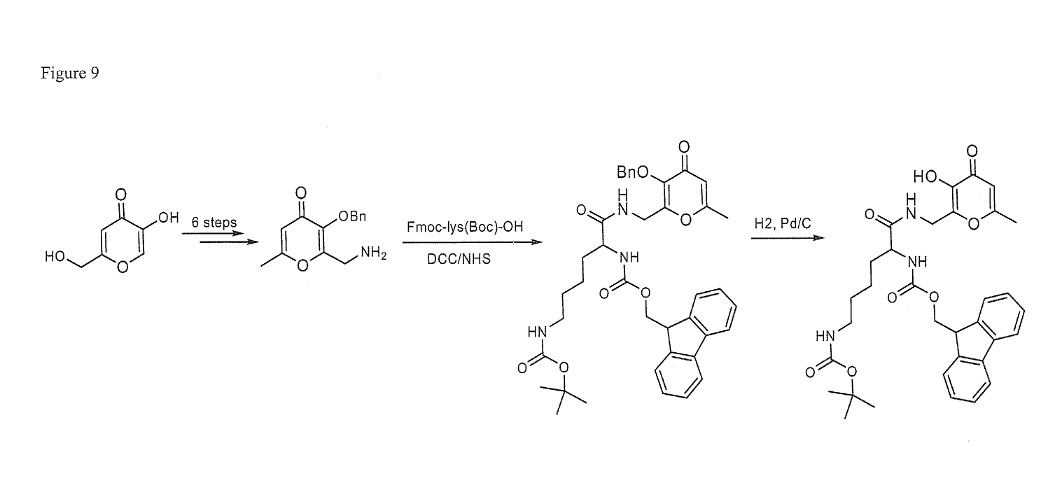

The synthesis of bidentate hydroxypyranone is shown in FIG. 9. The synthesis of 2-aminomethyl-6-methyl-3-benzyloxy-pyran-4-one has also been reported in the same paper indicated above. Similarly, this amine was coupled with activated Fmoc-lys(Boc)-OH and followed by hydrogenation to afford the desired bidentate hydroxypyranone.

iii) Hexadentate Hydroxypyridinone

The synthesis of hexadentate hydroxypyridinone is shown in FIG. 10. The benzyl protected tri-hydroxypyridinone containing a free amino group at one end was synthesized by our group previously [Zhou T. et al. J Med Chem. 2006, 49, 4171-4182]. An analogous two-step procedure gave the desired hexadentate hydroxypyridinone.

### iv) Hexadentate Hydroxypyranone

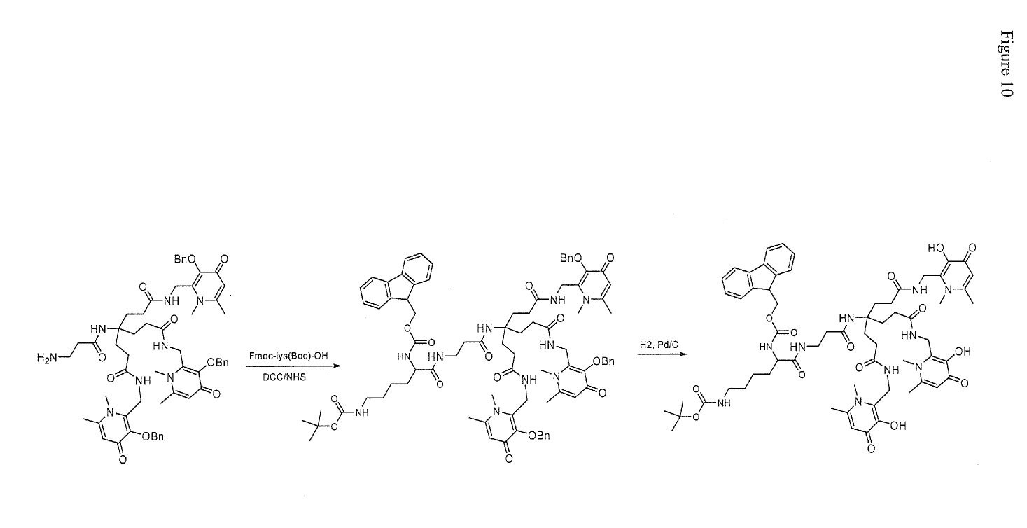

The synthesis of hexadentate hydroxypyranone is shown in FIG. 11. The synthesis of benzyl protected tri-hydroxypyranone containing a free amino group at one end has not been reported previously. However, in similar fashion to the synthesis of the hydroxypyridinone analogue, this compound can be produced from three portions of 2-aminomethyl-6-methyl-3-benzyloxy-pyran-4-one being coupled to the corresponding triacid. The resulting tripyranone amine was then coupled to the amine-protected lysine, followed by hydrogenation to afford the desired hexadentate hydroxypyranone.

### Example 7

### Coupling of Iron Chelators to Probe and Beads

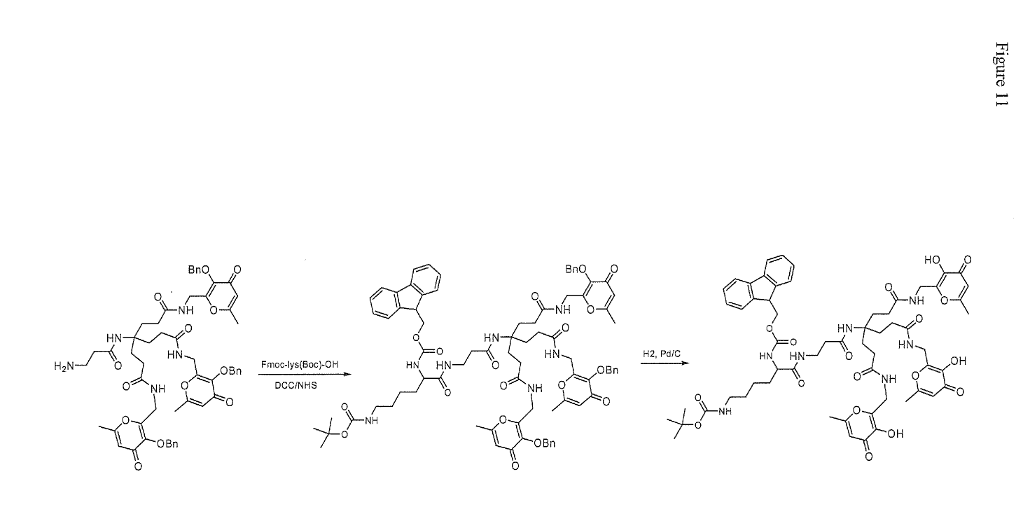

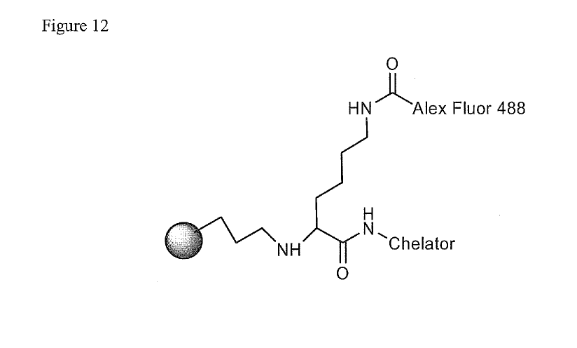

5 μmol of each iron chelator as described in Example 6 was weighed in an eppendorf tube. Dichloromethane (400 μl) and trifluoroacetic acid (200 μl) were added and the mixture was shaken at room temperature for 4 h. After removal of the solvent by vacuum, the residue was dissolved in dimethylformamide (200 μl). Alexa Fluor 488 (NHS activated) (1.25 mg) and triethylamine (10 μl) was added and the mixture was shaken overnight at room temperature. The solvent was evaporated again by high vacuum and the residue was added to 20% piperidine in DMF (200 μl) and shaken for 3 h. After removal of piperidine by high vacuum, the residue was dissolved in DMF (200 μl) and the solution was added to Dynabeads M-280 tosylactivated (200 μl, 2×10⁹ beads/ml, pre-washed by PBS buffer three times) and incubated at 37° C. overnight. The labeled beads were then washed with PBS buffer (3 times) and diluted with PBS to 5×10⁶ beads/ml. The beads were stored in fridge and are ready for use. The general structure of the beads is shown in FIG. 12.

### Example 8

### Preparation of Standard Curves of the Four Beads

1 mM Fe-NTA solution was prepared from iron atomic absorption standard solution (1008 mg/1, 56 μl) and 2.5 equiv. NTA (100 mM, 25 μl) in water (919 μl). 32 μM Fe-NTA solution was prepared by taking 32 μl 1 mM Fe-NTA with 968 μl water. Fe solutions at 16, 8, 4, 2, 1, 0.5 μM respectively were prepared by subsequent dilutions of 1:1 in water from 32 μM. 20 μl of probe labeled beads as described in Example 7 (5×10⁶ beads/ml) were placed in a 96 well plate, followed by 160 μl PBS buffer and various concentrations of Fe-NTA (20 μl). All the samples were prepared in duplicate. The mixtures were incubated for 30 min before measuring fluorescence.

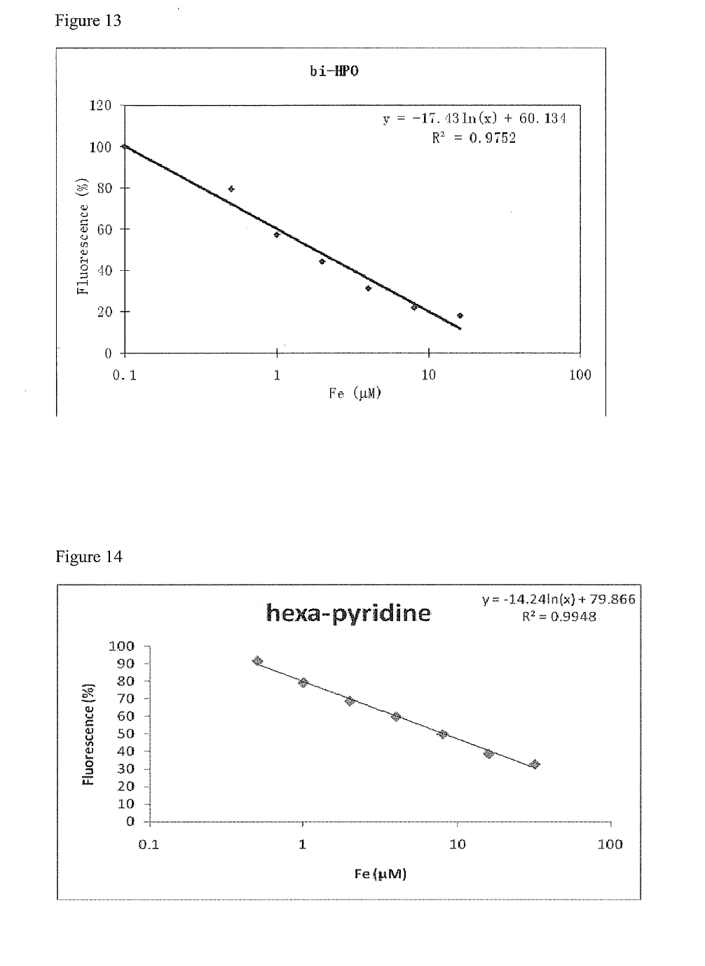

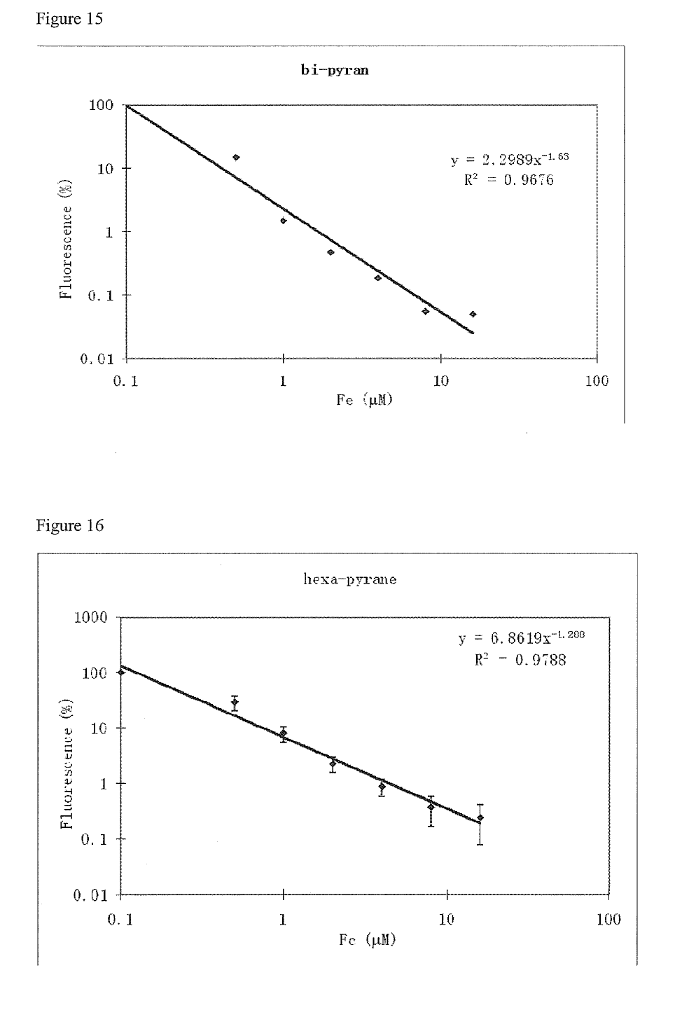

Fluorescence measurements were carried out on a Beckman Coulter FC500 flow cytometer. Median fluorescence of at least 10,000 events or at least 5 min scanning time were recorded and corrected for cell auto-fluorescence. X-median 50 values were calculated for medians of three independent experiments. The standard curves of these beads are presented in FIGS. 13 to 16. All four preparations provide sensitivity to iron over the concentration range 0.1-16 μM.

### Example 9

Standard Curve with the Albumin-Attached Fluorescent Beads

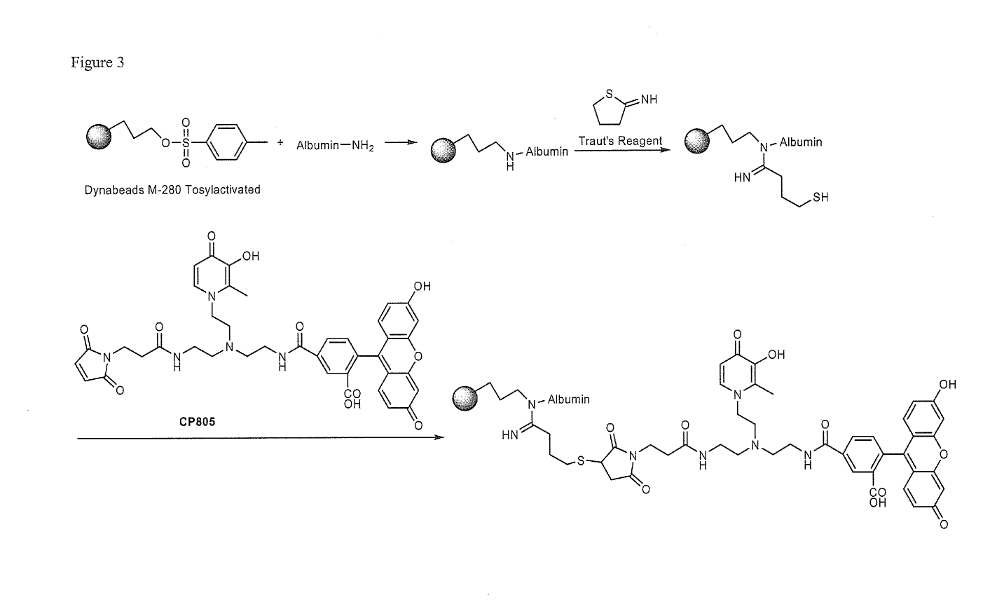

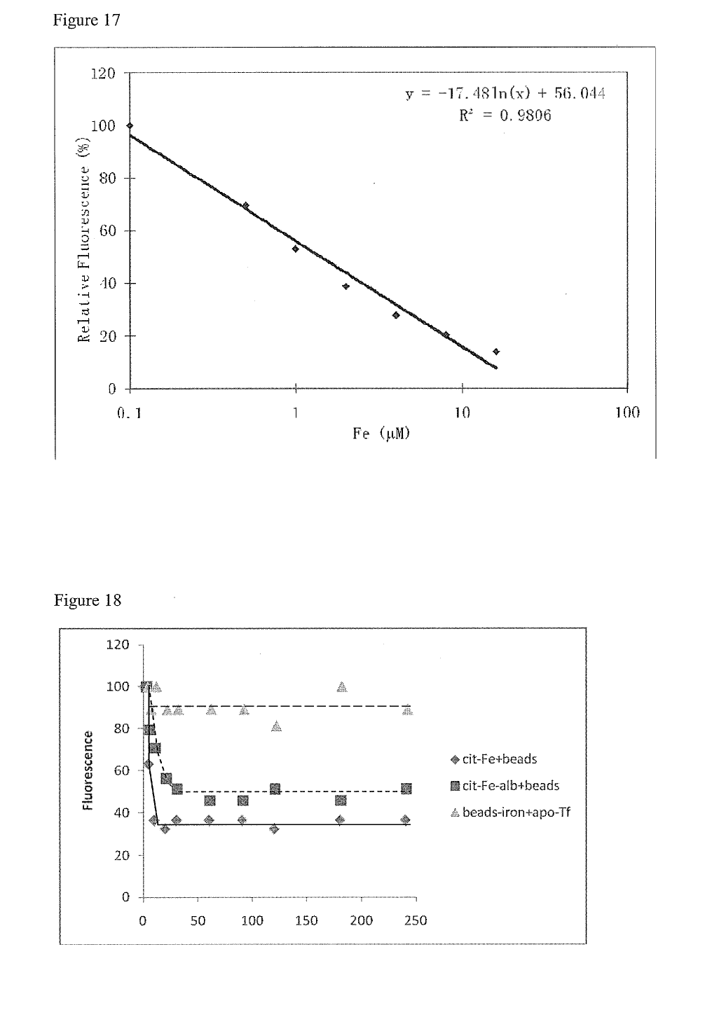

For comparison, fluorescent beads conjugated to CP805 via albumin as shown in FIG. 3 were prepared, as described in Examples 1 and 2. A standard curve was prepared for these beads using the method of Example 8, and is shown in FIG. 17. The sensitivity of the CP805-albumin conjugated beads is similar to that of the hydroxypyridinone and hydroxypyranone beads of Examples 6 to 8.

### Example 10

Transfer of Iron from Ferritin, Transferrin and Simulated Non-Transferrin-Bound Iron

A solution of iron-citrate complex (1:10 molar ratio, 10 μM based on iron concentration) was investigated with all four sets of beads described in Example 7. After 30 min incubation in the presence of the fluorescent beads, the fluorescence was monitored and the detectable iron concentration was calculated based on the relevant standard curve.

The results are shown in Table 1 below. Beads coupled with hexadentate hydroxypyridinone showed the best results, detecting the majority of the available iron in the indicated incubation time. Thus hexadentate hydroxypyridinone beads were selected for further studies.

TABLE 1
Detected iron level of iron-citrate (10 μM:100 μM) by different
fluorescent beads

| | bidentate | bidentate | hexadentate | hexadentate |
|---|---|---|---|---|
| | hydroxy- | hydroxy- | hydroxy- | hydroxy- |
| Bead type | pyridinone | pyranone | pyridinone | pyranone |
| Fe | 3.97 ± 1.02 | 0.45 ± 0.24 | 9.35 ± 1.20 | 1.61 ± 0.50 |
| concentration | | | | |
| (μM) | | | | |

Solutions containing physiological concentrations of ferritin (100 μg/1), mono-iron transferrin (32.5 μM), iron-albumin complexes (1 μM:40 g/l, overnight pre-incubation), iron-citrate complexes (1:10 ratio, 1 and 10 μM respectively, based on iron concentration, incubated overnight), iron-citrate-albumin complexes (1 μM:10 μM:40 g/l, incubated overnight) and iron-NTA complex (1 μM:8 mM and 1 μM:80 mM respectively) were investigated with the hexadentate hydroxypyridinone beads. After 30 min incubation of the fluorescent beads with the above solutions, the fluorescence was monitored and the iron level was calculated (Table 2). With the ferritin and mono-transferrin solutions the levels of iron detected were less than 0.2 μM, which indicates that there was little iron extracted from these iron proteins.

When either 8 mM or 80 mM NTA was incubated with iron (1 μM), the iron level detected by the hexadentate hydroxypyridinone beads was similar, which indicated that the presence of NTA even in a large excess did not influence the chelation of the beads with iron. Over 80% of iron was recovered from other ligands such as citrate (can combine with iron to form either mono-nucleic or oligomeric iron) and albumin. When iron was incubated with citrate and albumin overnight, a slightly lower scavenging efficiency was recorded but over 70% of the available iron was detected (Table 2).

TABLE 2
Detected iron level from different iron ligands by hexadentate
hydroxypyridinone beads

| ligand | Iron level |
|---|---|
| Ferritin (100 μg /l) | 0.14 ± 0.13 |
| mono-transferrin (32.5 μM) | 0.08 ± 0.02 |
| Fe-nta (1 μM:8 mM) | 0.89 ± 0.1 |
| Fe-nta (l μM:80 mM) | 0.89 ± 0.1 |
| Fe-cit (1 μM:10 μM) | 0.96 ± 0.19 |
| Fe-cit (1 μM:100 μM) | 0.98 ± 0.15 |
| Fe-alb (1 μM:40 g/l) | 0.82 ± 0.15 |
| Fe-cit-alb (1 μM:10 μM:40 g/l) | 0.73 ± 0.15 |

### Example 11

Kinetic Study of Hexadentate Hydroxypyridinone Beads with Iron-Citrate, Iron-Citrate-Albumin and Apo-Transferrin

Iron-citrate solution was prepared to final concentration of 10 μM iron with 100 μM citrate incubated overnight; iron-citrate-albumin solution was prepared by incubating iron (final at 10 μM) with citrate (final at 100 μM) overnight then 4 h incubation with albumin (final at 40 g/l); Apo-transferrin solution was prepared at 32.5 μM at final concentration. After mixing with beads, the fluorescence was monitored immediately and followed by at 5, 10, 20, 30, 60, 90, 120 180 and 240 min respectively. The results are presented in FIG. 18. The data demonstrated that in the presence of apo-transferrin, the fluorescence of the iron-beads complex (pre-prepared using same amount of beads with 10 μM iron) was stable over the 4-hour period, which indicated that the beads did not donate iron to apo-transferrin. However, in the presence of the other two solutions, the fluorescence was dramatically decreased in first 30 min and then leveled off, which indicated that 30 min was sufficient to scavenge the loosely available iron. It should be noted that with iron-citrate complex, the decrease in fluorescence of the beads was greater than that induced by the iron-citrate-albumin complex This observation agrees with the data in Table 2 indicating that there is more iron detected in the presence of the iron-citrate complex compared with the iron-citrate-albumin complex.

### Example 12

### Correlation Between the ‘Bead’ Method and the NTA Method for NTBI Measurements

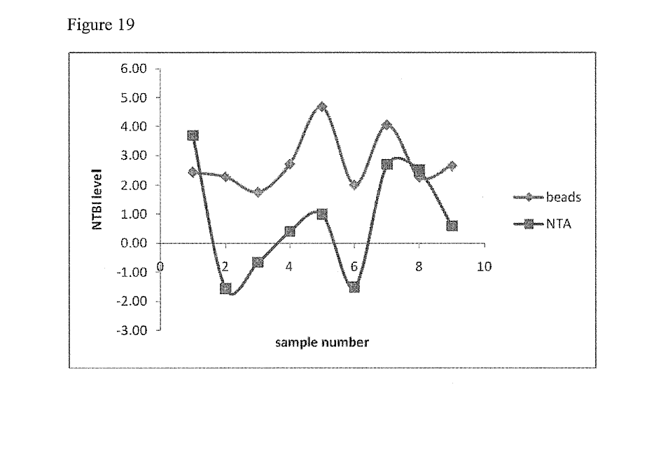

We made a comparison study between the hexadentate hydroxypyridinone ‘bead’ method and the established nitrilotriacetic acid (NTA) assay method. 9 serum samples from thalassaemia patients were collected from University College London and the NTA method was undertaken independently and the results were withheld until completion of the study. The results from two methods are shown in FIG. 19. The iron levels detected by the ‘bead’ method are about 2-3 μM higher than those obtained by the NTA method in most cases, which is possibly due to some fractions of the labile iron pool not being available to NTA. Generally there is the same trend observed for both methods. This indicates that the bead method is much more sensitive than the NTA assay, and is capable of accurately detecting lower NTBI concentrations.

### CONCLUSION

Hexadentate pyridinone linked beads have potential to scavenge all NTBI pools and do not exchange iron with ferritin and transferrin in the prescribed 30 min incubation time.

All publications mentioned in the above specification are herein incorporated by reference. Various modifications and variations of the described compounds and methods of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and related fields are intended to be within the scope of the following claims.

1. A fluorescent iron-binding compound bound to a solid phase.

2. The compound according to claim 1, comprising an iron-binding moiety and a fluorophore, wherein a fluorescent signal generated by the fluorophore is modulated in response to binding of iron to the iron-binding moiety.

3. The compound according to claim 1, comprising a group of formula I: 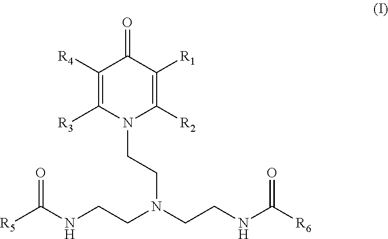
wherein:
R₁, R₂, R₃ and R₄ are each independently selected from hydrogen, hydroxyl and C₁-C₅ alkyl, e.g. methyl, provided that at least one of R₁ and R₄ is hydroxyl;
R₅ comprises a linkage to a solid phase; and
R₆ comprises a fluorophore.

4. The compound according to claim 1, comprising a fluorophore selected from the group consisting of fluorescein, rhodamine, dansyl, phycoerythrin, phycocyanin, allophycocyanin, o-phthaldehyde, cascade blue, coumarin, naphthalenes, pyrenes, pyridyloxazole derivatives and fluorescamine.

5. The compound according to claim 4, wherein the fluorophore comprises fluorescein.

6. The compound according to claim 1, which is covalently bound to the solid phase.

7. The compound according to claim 1, which is linked to a solid phase by a group of formula Ia: 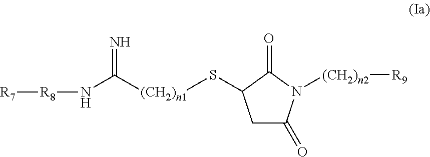
wherein R₇ comprises a solid phase; R₈ is optionally present and comprises a protein, R₉ comprises a bond to the compound, and n1 and n2 are each independently an integer between 1 and 5.

8. The compound according to claim 1, wherein the solid phase comprises heads or microspheres.

9. A method for detecting non-transferrin bound iron in a sample, comprising:
a) contacting the sample with a fluorescent iron-binding compound bound to a solid phase; and
b) detecting a fluorescent signal derived from the fluorescent iron-binding compound bound to the solid phase, wherein the fluorescent signal is indicative of non-transferrin bound iron levels in the sample.

10. The method according to claim 9, wherein the fluorescent signal is detected by flow cytometry.

11. The method according to claim 9, wherein the method provides a fluorescent signal indicative of non-transferrin bound iron levels at least in the concentration range 0.01 to 1 μM.

12. A method for monitoring iron overload in a subject, comprising detecting non-transferrin bound iron in a sample from the subject by a method according to claim 9.

13. A composition comprising a fluorescent iron-binding compound according to claim 1, wherein the composition is suitable to detect non-transferrin bound iron in a sample.

14. A kit for detecting non-transferrin bound iron in a sample, comprising a fluorescent iron-binding compound according to claim 1, packaged in one or more containers with one or more further reagents.

15. A compound according to formula (II): 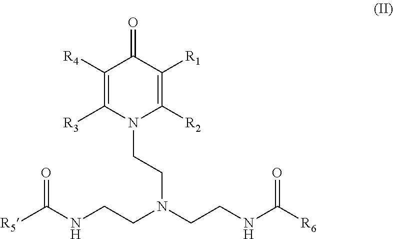
wherein R₁, R₂, R₃ and R₄ are each independently selected from hydrogen, hydroxyl and C₁-C₅ alkyl, e.g. methyl, provided that at least one of R₁ and R₄ is hydroxyl;
R₅′ comprises a group capable of forming a linkage to a solid phase; and
R₆ comprises a fluorophore.

16. A method for producing the compound of claim 3 comprising reacting a compound of formula (II) with a solid phase, wherein the compound of formula (II) is: 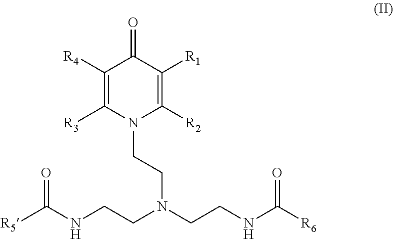
wherein R₁, R₂, R₃ and R₄ are each independently selected from hydrogen, hydroxyl and C₁-C₅ alkyl, e.g. methyl, provided that at least one of R₁ and R₄ is hydroxyl;
R₅′ comprises a group capable of forming a linkage to a solid phase; and
R₆ comprises a fluorophore.

17. The compound according to claim 1, comprising a group of formula VIII: 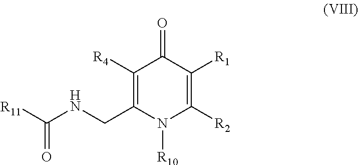
wherein:
R₁, R₂ and R₄ are each independently selected from hydrogen, hydroxyl and C₁-C₅ alkyl, e.g. methyl, provided that at least one of R₁ and R₄ is hydroxyl;
R₁₀ is hydrogen or C₁-C₅ alkyl, e.g. methyl; and
R₁₁ comprises a fluorophore and a linkage to a solid phase.

18. The compound according to claim 17, comprising a group of formula IX: 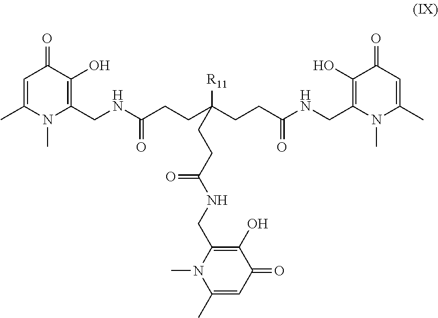
wherein R₁₁ comprises a fluorophore and a linkage to a solid phase.

19. The compound according to claim 1, comprising a group of formula XI: 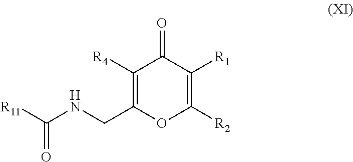
wherein:
R₁, R₂ and R₄ are each independently selected from hydrogen, hydroxyl and C₁-C₅ alkyl, e.g. methyl, provided that at least one of R₁ and R₄ is hydroxyl; and
R₁₁ comprises a fluorophore and a linkage to a solid phase.

20. The compound according to claim 19, comprising a group of formula XII: 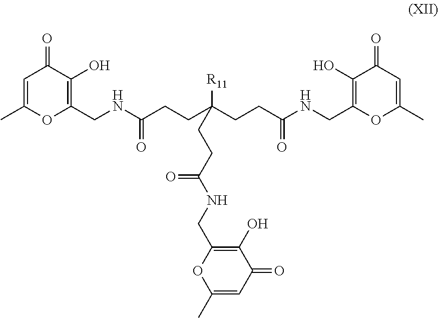
wherein R₁₁ comprises a fluorophore and a linkage to a solid phase.

21. The compound according to claim 17, wherein R₁₁ comprises a group of formula X: 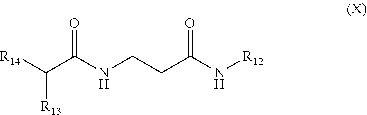
wherein R₁₂ comprises a bond to the compound of formula IX or formula XII;
R₁₃ comprises a fluorophore; and
R₁₄ comprises a linkage to a solid phase.

22. The compound according to claim 19, wherein R₁₁ comprises a group of formula X: 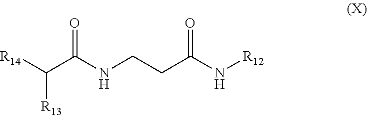
wherein R₁₂ comprises a bond to the compound of formula IX or formula XII;
R₁₃ comprises a fluorophore; and
R₁₄ comprises a linkage to a solid phase.
